SEQUENCE LISTING (18 sequences)

SEQ ID NO: 1 - DNA, 17 bp, Arabidopsis thaliana
  source 1..17: /organism=“Arabidopsis thaliana” /mol_type=“unassigned DNA”
ctgcttctcgctgctct

SEQ ID NO: 2 - DNA, 17 bp, Arabidopsis thaliana
  source 1..17: /organism=“Arabidopsis thaliana” /mol_type=“unassigned DNA”
agagcagcgagaagcag

SEQ ID NO: 3 - DNA, 18 bp, Arabidopsis thaliana
  source 1..18: /organism=“Arabidopsis thaliana” /mol_type=“unassigned DNA”
cttctgtttccacggcgg

SEQ ID NO: 4 - DNA, 18 bp, Arabidopsis thaliana
  source 1..18: /organism=“Arabidopsis thaliana” /mol_type=“unassigned DNA”
ccgccgtggaaacagaag

SEQ ID NO: 5 - DNA, 18 bp, Arabidopsis thaliana
  source 1..18: /organism=“Arabidopsis thaliana” /mol_type=“unassigned DNA”
catgcttgatgcgttgaa

SEQ ID NO: 6 - DNA, 18 bp, Arabidopsis thaliana
  source 1..18: /organism=“Arabidopsis thaliana” /mol_type=“unassigned DNA”
ttcaacgcatcaagcatg

SEQ ID NO: 7 - DNA, 18 bp, Arabidopsis thaliana
  source 1..18: /organism=“Arabidopsis thaliana” /mol_type=“unassigned DNA”
ttccacattaagtcccaa

SEQ ID NO: 8 - DNA, 18 bp, Petunia x hybrida
  source 1..18: /organism=“Petunia x hybrida” /mol_type=“unassigned DNA”
catgcttggtgccttgaa

SEQ ID NO: 9 - DNA, 18 bp, Petunia x hybrida
  source 1..18: /organism=“Petunia x hybrida” /mol_type=“unassigned DNA”
ttcaaggcaccaagcatg

SEQ ID NO: 10 - DNA, 18 bp, Petunia x hybrida
  source 1..18: /organism=“Petunia x hybrida” /mol_type=“unassigned DNA”
catgcttggggccttgga

SEQ ID NO: 11 - DNA, 18 bp, Petunia x hybrida
  source 1..18: /organism=“Petunia x hybrida” /mol_type=“unassigned DNA”
tccaaggccccaagcatg

SEQ ID NO: 12 - DNA, 17 bp, Arabidopsis thaliana
  source 1..17: /organism=“Arabidopsis thaliana” /mol_type=“unassigned DNA”
gggtatttcacattgtc

SEQ ID NO: 13 - DNA, 17 bp, Arabidopsis thaliana
  source 1..17: /organism=“Arabidopsis thaliana” /mol_type=“unassigned DNA”
gcttcaattgcagcaac

SEQ ID NO: 14 - DNA, 18 bp, Arabidopsis thaliana
  source 1..18: /organism=“Arabidopsis thaliana” /mol_type=“unassigned DNA”
cttctatttccgcggcgg

SEQ ID NO: 15 - DNA, 18 bp, Arabidopsis thaliana
  source 1..18: /organism=“Arabidopsis thaliana” /mol_type=“unassigned DNA”
ctcctattcccgcgacgg

SEQ ID NO: 16 - DNA, 18 bp, Nicotiana tabacum
  source 1..18: /organism=“Nicotiana tabacum” /mol_type=“unassigned DNA”
catgcttggcgcgttgaa

SEQ ID NO: 17 - DNA, 18 bp, Nicotiana tabacum
  source 1..18: /organism=“Nicotiana tabacum” /mol_type=“unassigned DNA”
catgcttggtgcattgaa

SEQ ID NO: 18 - DNA, 17 bp, Petunia x hybrida
  source 1..17: /organism=“Petunia x hybrida” /mol_type=“unassigned DNA”
ctccttcttgctgcctt

(19) United States
(12) Patent Application Publication
(10) Pub. No.: US 2016/0278382 A1
(43) Pub. Date: Sep. 29, 2016

(54) Usage of Oligonucleotides in Plant Biology

(71) Applicants: Chuanxin SUN, Uppsala (SE); Christer JANSSON, Walnut Creek, CA (US)

(72) Inventors: Chuanxin Sun, Uppsala (SE); Christer Jansson, Walnut Creek, CA (US)

(21) Appl. No.: 14/364,321

(22) PCT Filed: Dec. 6, 2012
(86) PCT No.: PCT/SE2012/051349

§ 371 Date: Jun. 11, 2014

(30) Foreign Application Priority Data
Dec. 12, 2011 (SE) 1151181-3

(51) Int. Cl. A01N 43/90; C12N 15/82

(52) U.S. Cl. CPC A01N 43/90; C12N 15/8218

(57) ABSTRACT

The invention relates to a method of producing transgenic plant material by transforming a plant with a vector comprising an essential gene having mutations at two sites at least. The method is exemplified with EPSPS as the essential gene. The method makes it possible to use an antisense molecule directed to the native form of said gene for selection of transformed plants. The application relates further to a plant obtainable by the method, a binary vector system containing the mutated essential gene, said mutations being silent mutations. Furthermore, the application discloses the use of an antisense molecule directed to an essential gene as a herbicide in particular using an aqueous solution comprising a saccharide such as sucrose, fructose and glucose.

### TECHNICAL FIELD

The present embodiments generally relate to the usage of oligonucleotides in plant biology, and in particular to the usage of such oligonucleotides in the formation of transgenic plant materials and as herbicide.

### BACKGROUND

Transgenic plants are generated by altering the genetic makeup of their genome using various genetic engineering techniques. Today common such engineering approaches include biolistic methods and Agrobacterium tumefaciens mediated transformation.

Briefly, in a biolistic method DNA bound to tiny particles are shot into plant tissue or single plant cells using a particle gun. The particles thereby penetrate the cell wall and the cell membranes to deliver the DNA in the plant cells where it becomes integrated into the genome of the plant cell.

Agrobacteria are natural plant bacteria that insert their genes into plant hosts, causing proliferation of plant cells near the soil level and the crown gall disease. The genetic information required for the proliferation is encoded on a mobile plasmid. When Agrobacterium infects a plant, it transfers this T-DNA to a random site in the plant genome. In the Agrobacterium tumefaciens mediated transformation the bacterial T-DNA is removed from the plasmid and is replaced with genetic material to be introduced into the plant.

A limitation with the prior art engineering techniques to produce genetically modified plants, such as exemplified above, is the usage of marker genes that are employed to identify the plants into which the genetic material has been incorporated. Today, such marker genes often provide resistance to antibiotics or herbicides. However, it is generally preferred to form marker-free transformants lacking the antibiotic or herbicide resisting genes. Such marker-free transformants can be obtained by removing the marker genes after transformation by different methods, including segregation after co-transformation, recombination-mediated deletion and transposon-based deletion, see Darbani et al., 2007. These methods are, though, tremendously time-consuming.

An alternative approach uses markers like phosphomannose isomerase genes, Sonntag et al., 2004, and D-amino acid oxidase genes, Erikson et al., 2004. However, even in these approaches foreign functions and proteins are still required and introduced into the plant cells, with a risk of new traits being spread to other species through horizontal and vertical gene-transferring flows.

There is therefore a need of an approach to generate transgenic plants that provides truly safe selection methods for identifying successfully transformed plants.

### SUMMARY

A general objective of the embodiments is directed towards the usage of oligonucleotides in plant biology.

A particular objective relates to the usage of antisense oligonucleotides in producing transgenic plant material.

Another particular objective relates to the usage of antisense oligonucleotides as herbicides.

These and other objectives are met by embodiments disclosed herein.

An aspect of the embodiments defines a method of producing transgenic plant material. The method comprises transforming a first plant material with a vector comprising a gene of interest that is capable of being transcribed in the first plant material and/or in a second plant material generated from the first plant material and a mutated essential gene that is capable of being transcribed in the first plant material and/or in the second plant material. The mutated essential gene encodes a molecule that is essential for the survival of the first plant material and/or the second plant material and comprises at least two site mutations with regard to a native form of the essential gene encoding the molecule and present in the first plant material and/or the second plant material. The method further comprises contacting the first plant material or the second plant material with an antisense oligonucleotide capable of hybridizing to a portion of mRNA transcribed from the native form of the essential gene, thereby inhibiting its translation. This portion of mRNA is transcribed from a portion of the essential gene encompassing at least two nucleotides that are site-mutated in the mutated essential gene. The first plant material or the second plant material is then identified as transgenic plant material capable of transcribing the gene of interest if the first plant material or the second plant material does not show symptoms of gene inhibition with regard to the essential gene.

A related aspect of the embodiments defines a transgenic plant material obtainable according to the method described above.

A further related aspect of the embodiments relates to a binary vector system comprising a mini-Ti plasmid comprising an origin for replication for Agrobacterium, a gene of interest that is capable of being transcribed in a plant material and a mutated essential gene that is capable of being transcribed in the plant material. The mutated essential gene encodes a molecule that is essential for survival of the plant material and comprises at least two silent site mutations with regard to a native form of the essential gene present in the plant material and encoding the molecule. The molecule encoded by the mutated essential gene preferably has an identical amino acid sequence as the molecule encoded by the native form of the essential gene. The gene of interest and the mutated essential gene are inserted into a T-DNA region of the mini-Ti plasmid. The binary vector system also comprises a helper Ti plasmid lacking the T-DNA region but comprising a vir region.

Another aspect of the embodiments relates to the use of an antisense oligonucleotide capable of hybridizing to a portion of mRNA transcribed from an essential plant gene as herbicide.

Another related aspect of the embodiments defines a method of killing a plant by contacting the plant with an aqueous solution comprising an antisense oligonucleotide capable of hybridizing to a portion of mRNA transcribed from an essential plant gene of the plant. The aqueous solution also comprises at least one saccharide selected from the group of sucrose, fructose and glucose.

A further related aspect of the embodiments defines a herbicide composition comprising an aqueous solution of an antisense oligonucleotide capable of hybridizing to a portion of mRNA transcribed from an essential plant gene of a plant and at least one saccharide selected from the group of sucrose, fructose and glucose.

### BRIEF DESCRIPTION OF THE DRAWINGS

The invention, together with further objects and advantages thereof, may best be understood by making reference to the following description taken together with the accompanying drawings, in which:

FIG. 1 illustrates the results of antisense oligodeoxynucleotide (ODN) inhibition in plant germination. An antisense ODN against the conserved region of the gene encoding 5-enolpyruvylshikimate-3-phosphate synthase (EPSPS) in Arabidopsis with two mismatches to the tobacco EPSPS1 and EPSPS2, respectively, inhibited Arabidopsis but not tobacco germination (a). A mixture of antisense ODNs against the petunia EPSPS1 and EPSPS2 with two and four mismatches to the Arabidopsis EPSPS inhibited petunia but not Arabidopsis germination (c). The corresponding sense ODNs against the same regions did not cause inhibition of germination (b, d).

FIG. 2 illustrates antisense ODN inhibition in plant leaves. An independent ODN against Arabidopsis EPSPS was used for inhibition of leaf growth. The leaves were incubated with 700 μM ODN in 80 mM sucrose for 48 hours. ODN inhibition was found in the Arabidopsis leaves with antisense ODN, but not in petunia. Inhibition was not observed in the control (At EPSPS sense ODN).

FIG. 3 illustrates antisense ODN inhibition in dicotyledons (dicots) (Arabidopsis). Two antisense ODNs against the gene encoding 1-deoxy-D-xylulose-5-phosphate reductoisomerase (DXR) were used for inhibition of seed germination. Germination was conducted in a modified Murashige and Skoog (MS) medium with 400 μM ODN for Arabidopsis. ODN inhibition was found in the two antisense ODN in Arabidopsis (a, b) but not in the control (c).

FIG. 4 illustrates antisense ODN inhibition in monocotyledons (monocots) (rice). Two antisense ODNs against DXR were used for inhibition of seed germination. Germination was conducted in a modified MS medium with 1.6 mM ODN for rice. ODN inhibition was found for the antisense ODN in rice.

FIG. 5 is a schematic representation of a new concept on development of antisense ODN inhibition technology in creating a marker-free selection system. A plant with an endogenous essential gene, such as EPSPS (white dot on the chromosomes identified by white full arrows) is exposed to an antisense ODN that is 100% complementary to the EPSPS mRNA. The antisense ODN binds to the cognate mRNA and inhibits EPSPS synthesis. The plant dies due to the lack of EPSPS synthesis (upper panel). However, a plant with an EPSPS gene containing two mismatches to the antisense ODN (white dot on the chromosomes identified by white broken arrow) can avoid the antisense ODN binding and is able to survive under the antisense ODN selection pressure (lower panel).

FIG. 6 illustrates the results of screening of ODN. Four antisense ODNs were designed and tested for inhibition in Arabidopsis leaf experiments. A sense ODN was used as control.

FIG. 7 illustrates the At EPSPS antisense ODN2-binding region of Arabidopsis EPSPS (GeneBank accession no. NM_130093.2), which was selected as an essential gene for two nucleotide mutations. The two mutated nucleotides are A and G from G and A. The experimentally used sense and antisense ODN sequences (At EPSPS sense ODN2 and At EPSPS antisense ODN2) are also shown.

FIG. 8 illustrates results demonstrating that the constructed kit worked for Arabidopsis. Sense (a and b) and antisense (c and d) ODNs, respectively, were applied to wild type Arabidopsis (a and c) and transgenic Arabidopsis homozygotes with the two nucleotides modified EPSPS (b and d).

FIG. 9 is a schematic drawing of the construction of the binary vector using the two nucleotides mutated Arabidopsis EPSPS as a selectable marker gene. In the figure, LB indicates left border of T-DNA region, RB indicates right border of T-DNA region, nos t denotes nos terminator and 35S p denotes 35S promoter.

FIG. 10 illustrates the results of leaf experiments demonstrating two and five nucleotide mutations can escape the ODN inhibition. Leaves of wild type (a) and transgenic plants with 0-site mutated EPSPS showed an inhibition symptom (as indicated by the arrows) and 2-site (c) and 5-site (d) mutations remained healthy after 48 hours of ODN incubation.

FIG. 11 illustrates the results of using antisense ODNs as gene herbicides. At EPSPS antisense ODN2 were used for spraying seedlings of Arabidopsis at a concentration of 700 μM in 80 mM sucrose. Arabidopsis growth was inhibited and some of the plants were killed by At EPSPS antisense ODN2 as indicated by the arrow. At EPSPS sense ODN2 was used as a control.

### DETAILED DESCRIPTION

The present embodiments generally relate to the usage of oligonucelotides in plant biology, and in particular to the usage of oligonucleotides in producing transgenic plants and plant materials and as biological herbicides.

An oligonucleotide is a short nucleic acid polymer typically with fifty or fewer nucleotides. The oligonucleotides of the embodiments could consist of naturally occurring nucleotides, i.e. deoxyribonucleotides or ribonucleotides. In the former case, oligonucleotides composed of 2′-deoxyribonucleotides, i.e. fragments of DNA, are used and are generally denoted oligodeoxynucleotides (ODNs). Oligoribonucleotides (ORNs) are instead composed of ribonucleotides, i.e. fragments of RNA. The embodiments are, however, not limited to the usage of oligonucleotides composed of naturally occurring nucleotides, i.e. ODNs or ORNs. Also oligonucleotides that are composed of artificial nucleotides could be used as long as they are capable of binding and hybridizing to a portion of mRNA transcribed from an essential plant gene as disclosed herein. Non-limiting example of such artificial nucleotides includes oligonucleotides with methylphosphonates, phosphorothioates and peptide nucleic acid (PNA). Such artificial nucleotides can be used to increase the stability of the oligonucleotides and/or their efficiency to enter plant cells. The embodiments in particular use ODNs.

The oligonucleotides of the present embodiments are up 50 nucleotides long and preferably from about 12 nucleotides to about 40 nucleotides, in particular 12-30 nucleotides, preferably 15-25 nucleotides or 15-20 nucleotides, such as about 17 or 18 nucleotides. A particular example of oligonucleotides of the embodiments is an ODN having a length of 15-25 nucleotides, such as 15-20 nucleotides.

The oligonucleotides of the embodiments are antisense oligonucleotides, i.e. single strands of nucleotides that are complementary to and bind to a target sequence. An antisense ODN targets a specific, complementary coding RNA by binding to this RNA molecule and causing, in a plant cell, degradation of the RNA molecule through the action of the enzyme RNase H that degrades DNA/RNA hybrids. Correspondingly, an antisense ORN targets a specific, complementary coding RNA by binding to this RNA molecule and thereby preventing protein translation of the RNA molecule by the ribosomes in a plant cell.

An aspect of the embodiments relates to the usage of oligonucelotides in the formation of transgenic plant material.

An embodiment of this aspect defines a method of producing transgenic plant material. As used herein plant material denotes any plants or material or tissue obtained from plants. Plant material thereby encompasses plant cells, plant cell resuspension culture, complete plants or parts of plants, plant tissue, seeds, plant tissue cultures, such as embryo or callus.

The method comprises transforming a first plant material with a vector comprising i) a gene of interest that is capable of being transcribed in the first plant material and/or in a second plant material generated from the first plant material and ii) a mutated essential gene that is capable of being transcribed in the first plant material and/or in the second plant material.

The second plant material could be any plant material, such as a plant or plant tissue, which is generated, directly or indirectly, from the first plant material, such as a seed, plant cells, plant or plant tissue culture. Thus, if the vector is used to introduce a gene of interest in the first plant material any second plant material that is generated from or obtained based on the first plant material has the gene of interest incorporated therein in such a way that the gene of interest is capable of being transcribed in the second plant material. In a particular embodiment, the gene of interest is thereby incorporated into the genome of the second plant material.

The gene of interest can be any gene that one would like to introduce and express in plant material to form genetically modified plant material, i.e. transgenic plant material. The embodiments are not limited to any particular gene of interest. The gene of interest can come from the same kingdom, i.e. from another plant species, or from another kingdom, such as from bacteria. The gene of interest encodes a certain functional molecule, such as polypeptide or protein, that can give a desired characteristic or function to the plant material. Examples include resistance to abiotic and biotic stresses, such as pests, diseases or environmental conditions, or the production of any value-added materials, such as certain nutrients (including secondary metabolites, lipids, carbohydrates and proteins), pharmaceutical agents and/or biomass for biofuels

The vector not only comprises the gene of interest but also a mutated essential gene that is capable of being transcribed in the first plant material and/or in the second plant material. The mutated essential gene encodes a molecule that is essential for the survival of the first plant material and/or the second material. Thus, without production of the molecule the plant material will not be viable. For instance, lack of the molecule could be manifested in plant cell death, i.e. an “apoptosis-like” symptom. Alternatively, growth of the plant material can be inferior as compared to plant material in which the molecule is produced. Thus, lack of production of the molecule in the plant material can be clearly visibly verified and be used to discriminate between plant material in which the molecule is not being produced and plant material in which the molecule is produced.

This mutated essential gene is a mutated form of a native, i.e. non-mutated, form of the essential gene that naturally exists in the first plant material and/or in the second plant material. The mutated essential gene comprises at least two site mutations with regard to the native form of the essential gene encoding the molecule. This means that at least two of the nucleotides in the native or wild type form of the essential gene that naturally is present in the first plant material and/or the second plant material have been changed to other nucleotides. The at least two site mutations are furthermore present basically in the same region of the DNA sequence of the essential gene. This means that when an mRNA has been transcribed from the mutated essential gene at least two mutated ribonucleotides in the mRNA are at most spaced apart at a length in terms of nucleotides that is not longer than the length in terms of nucleotides of the oligonucleotide used in the production of the transgenic plant. Hence, if the oligonucleotide comprises, for instance, 20 nucleotides the at least two site mutations are positioned in the mutated essential gene so that the at least two mutated ribonucleotides in the mRNA transcribed from the mutated essential gene are spaced apart with at most 18 ribonucleotides. In a general embodiment, if the oligonucleotide comprises N nucleotides the at least two site mutations are positioned in the mutated essential gene so that the at least two mutated ribonucleotides in the mRNA transcribed from the mutated essential gene are spaced apart with at most N-2 ribonucleotides. Preferably, the at least two site mutations are positioned so that the resulting mutated ribonucleotides will not pairwise be separated with more than about five to ten ribonucleotides.

Generally, close proximity of the site mutations relative each other implies that they are typically positioned close to each other in the mutated essential gene, such as in the same exon, but could be present in neighboring exons if any intermediate intron is spliced away so that the mutated nucleotides are brought in close proximity to each other in the formed mRNA after splicing.

The at least two site mutations in the mutated essential gene with regard to the native form of the essential gene are preferably silent mutations. This means that the at least two site mutations preferably do not result in a change in the amino acid sequence of the polypeptide or protein that is encoded by the essential gene. The site mutations are therefore preferably synonymous mutations. This means that a site mutation causes a change in the codon in the essential gene to a new codon in the mutated essential gene. However, the codon and the new codon preferably both code for the same amino acid. As a result the molecule encoded by the mutated essential gene preferably has an identical amino acid sequence as the molecule encoded by the native form of the essential gene.

A next step in the method of producing transgenic plant material is to contact the first plant material or the second plant material generated from or based on the first plant material with an antisense oligonucleotide. This antisense oligonucleotide is capable of hybridizing and binding to a portion of the mRNA transcribed from the native form of the essential gene. This portion of the mRNA to which the antisense oligonucleotide can bind is transcribed from a portion of the essential gene that encompasses the at least two nucleotides that are site-mutated in the mutated essential gene.

This means that the antisense oligonucleotide is capable of hybridizing to a portion of the mRNA transcribed from the native form of the essential gene. However, the at least two site mutations in the mutated essential gene give rise to an mRNA to which the antisense oligonucleotide does not hybridize or at least bind to with a significantly lower binding strength as compared to the binding between the mRNA from the native form of the essential gene and the antisense oligonucleotide. This means that the hybrid formed between the antisense oligonucleotide and the mRNA will effectively prevent or at least significantly inhibit translation of the mRNA from the native form of the essential gene. However, the comparatively much lower binding strength between the antisense oligonucleotide and the mRNA from the mutated essential gene, due to at least two base-pair mismatches, is not sufficient to prevent translation of the mRNA from the mutated essential gene. Thus, the antisense oligonucleotide is capable of effectively inhibiting translation of the mRNA from the native form of the essential gene but is not capable of significantly inhibiting translation of the mRNA from the mutated essential gene.

In the above mentioned contacting step a single species of antisense oligonucleotides could be used, i.e. all antisense oligonucleotides have the same nucleotide sequence. Alternatively, a combination of multiple, i.e. at least two, different antisense oligonucleotides having different nucleotide sequences can be used. These different antisense oligonucleotides are then capable of hybridizing to portions of mRNA transcribed from the native form of the essential gene but do not hybridize or bind poorly, i.e. with a significantly lower binding strength as compared to the mRNA from the native essential gene, to mRNA from the mutated essential gene.

The method further comprises identifying the first plant material or the second plant material as transgenic plant material capable of transcribing the gene of interest if the first plant material or the second plant material does not show any symptoms of gene inhibition with regard to the essential gene.

Thus, a plant material that is viable indicates that the plant material is producing the particular molecule that is encoded by the native form of the essential gene and the mutated essential gene. This further implies that the plant material has incorporated the mutated essential gene and thereby also the gene of interest as these two were present in the same vector. The plant material therefore has two independent sources for producing the molecule, i.e. the native form of the essential gene and the introduced mutated essential gene. The addition of the antisense oligonucleotides substantially shuts down the native form of the essential gene by hybridizing to its mRNA. However, since the plant material also has the mutated essential gene it can still produce the relevant molecule and is therefore viable. The presence of the mutated essential gene, which is obvious from the lack of gene inhibition and viability of the plant material, is an indication or selective marker that the plant material also incorporates the gene of interest since this was introduced together with the mutated essential gene.

However, a plant material lacking the gene of interest will also lack the mutated essential gene. When the plant material is contacted with the antisense oligonucleotide, the antisense oligonucleotide will prevent production of the relevant molecule from the native form of the essential gene. No such molecule or too low levels of this molecule is thereby produced by the plant material. This presents visible symptoms of gene inhibition such as in terms of inferior growth or cell death in the plant material. The particular symptom of gene inhibition depends on the essential gene and its gene product, i.e. the relevant molecule.

As is shown in the experiments presented herein two site mutations in the mutated essential gene with regard to the native form of the essential gene are sufficient to escape gene inhibition. However, the mutated essential gene may also comprise more than two site mutations, such as three, four, five or even more site mutations. However, it is generally sufficient to only include two site mutations but the embodiments work very well with more than two site mutations.

The selection of transgenic plant material according to the embodiments can be performed directly on the plant material which is transformed with the vector comprising the gene of interest and the mutated essential gene, i.e. the first plant material. Alternatively, the selection is performed on the second plant material, such as a complete plant, obtained or generated from the transformed first plant material.

Thus, this aspect of the embodiments is capable of producing transgenic plant material, such as transgenic plants, by the use of a mutated essential gene with at least two, preferably silent, site mutations together with antisense oligonucleotides, preferably antisense ODNs, as selection markers. No environmentally unfriendly substances, such as herbicides or antibiotics, are thereby needed in the selection process and the molecule encoded by the mutated essential gene is identical to the native form of the molecule. The antisense ODNs are biodegradable and will not have any environmental impact if used commercially.

A particular embodiment relates to a method of producing transgenic plant material. The method comprises transforming a first plant material with a vector comprising i) a gene of interest that is capable of being transcribed in at least one of the first plant material and a second plant material generated based on the first plant material and ii) a mutated essential gene that is capable of being transcribed in the at least one of the first plant material and the second plant material, wherein the mutated essential gene encodes a molecule that is essential for survival of the at least one of the first plant material and the second plant material and comprises at least two site mutations with regard to a native form of the essential gene encoding the molecule. The method also comprises contacting one of the first plant material and the second plant material with an antisense oligonucleotide capable of hybridizing to a portion of mRNA transcribed from the native form of the essential gene, the portion of mRNA is transcribed from a portion of the essential gene encompassing at least two nucleotides that are site-mutated in the mutated essential gene. The method further comprises identifying the one of the first plant material and the second plant material as transgenic plant material capable of transcribing the gene of interest if the one of the first plant material and the second plant material does not show symptoms of gene inhibition with regard to the native form of the essential gene.

As previously mentioned herein the gene of interest and the mutated essential gene are capable of being transcribed in the first plant material or the second plant material. This means that the vector preferably comprises a promoter operatively linked to the gene of interest and a promoter operatively linked to the mutated essential gene and where the promoters are active or can be induced to be active to thereby achieve transcription of the gene of interest and the mutated essential gene in the first plant material or the second plant material. The same type of promoter or different promoter types can be used for the gene of interest and the mutated essential gene. The promoters can be continuously transcriptionally active or can be inducible promoters in the plant material. Any promoter that is active or can be induced to be active in the relevant plant material can be used according to the embodiments. Non-limiting examples of such plant promoters include any constitutive promoters, stimuli-inducible promoters and tissue-specific promoters, such as 35S promoter, hormone/chemicals-inducible promoters and seed-specific promoters.

It is also possible to select and use the native promoter of the essential gene as present in the plant material as promoter for the mutated essential gene. In such a case, the mutates essential gene is typically regulated and transcribed in a same way as the essential gene when the plant material has been transformed with the vector comprising the native promoter of the essential gene operatively linked to the mutated essential gene and a promoter operatively linked to the gene of interest.

Also other regulatory elements can be present in the vector, such as terminators, for instance nos terminators.

The transforming step of the method can be performed according various known techniques that are capable of introducing foreign genetic material into plant materials. Thus, any such prior art technique that is capable of introducing a vector comprising the gene of interest and the mutated essential gene can be used in the method.

An example of such a method is the biolistic method that uses a particle gun to “shoot” the vector bound to tiny particles of, for instance, gold or tungsten into the plant material under high pressure. The accelerated particles thereby penetrate both the cell wall and the membranes. The vector then separates from the particles and is integrated into the plant genome inside the nucleous. The biolistic method has been successfully used to introduce gene of interests in especially monocotyledons (monocots).

Another example of a method to transform plant materials is to use Agrobacterium. An embodiment of such a technique involves introducing a mini-Ti plasmid, also denoted micro-Ti plasmid, wide-host-range small replicon or recombinant small replicon in the art, into an Agrobacterium cell, preferably an Agrobacterium tumefaciens cell. The mini-Ti plasmid comprises an origin for replication for Agrobacterium, the gene of interest and the mutated essential gene. The gene of interest and the mutated essential gene are inserted into a T-DNA region of the mini-Ti plasmid. The gene of interest and the mutated essential gene are thereby flanked by a left and a right T-DNA border or at least the right T-border.

The mini-Ti plasmid is introduced into an Agrobacterium cell comprising a helper Ti plasmid lacking the T-DNA region but comprising a vir (virulence) region. The helper Ti plasmid is generally present in the Agrobacterium cell when the mini-Ti plasmid with the gene of interest and the mutated essential gene is introduced into the Agrobacterium cell. This is, however, not necessary and the helper Ti plasmid can be introduced into the Agrobacterium cell prior to, simultaneously with or after introduction of the mini-Ti plasmid.

In a particular embodiment, the mini-Ti plasmid also has an origin for replication for Escheria coli. In such a case, the mini-Ti plasmid can be transferred from E. coli into Agrobacterium by i) a three way cross or ii) by direct transformation of an Agrobacterium strain containing the helper Ti plasmid. Alternatively, the mini Ti plasmid itself may be capable of conjugational transfer.

The first plant material is then transformed using the Agrobacterium cell carrying the mini-Ti plasmid and the helper Ti plasmid. The vir genes present in the helper Ti plasmid induce the transfer of T-DNA containing the gene of interest and the mutated essential gene of the mini-Ti plasmid into plant cells of the first plant material. The Agrobacetrium technique is in particular suitable for usage in connection with dicotyledons (dicots).

The first plant material can be transformed with the Agrobacetrium cell according to various techniques, such as by growing or culturing the first plant material in a culture medium comprising the Agrobacteria. A particular transforming technique that can be used is the so-called floral-dip method that involves dipping at least a portion of the first plant material in a solution comprising the Agrobacteria, see for instance Desfeux et al., 2000. For instance, plant materials, such as callus, can be inoculated with the Agrobacteria culture and then regenerate a new plant from the callus.

Contacting the transformed plant material with the antisense oligonucleotides can be performed according to various embodiments. In an embodiment, the first plant material or the second plant material is sprayed with an aqueous solution comprising the antisense oligonucleotide and at least one saccharide selected from the group of sucrose, fructose and glucose. The at least one saccharide present in the solution with the antisense oligonucelotide promotes uptake of the antisense oligonucleotide across the plant plasma membrane. This uptake is mediated by the active transport of mono- or disaccharides through sugar translocators as disclosed in Sun et al., 2007, 2008.

When spraying the aqueous solution onto the plant material, the antisense oligonucleotides will be taken up into plant cells of the plant material by the help of the added saccharide(s). The spraying technique is in particular suitable to use in connection with complete plants as the relevant plant material.

In a particular embodiment, the aqueous solution preferably comprises the antisense oligonucleotide at a concentration of at least 500 μM, preferably at least 750 μm. Also higher concentration intervals, such as at least 1000 μM, or concentrations lower than 500 μM can be used. Correspondingly, the total saccharide concentration in the aqueous solution to be sprayed is preferably at least 50 mM, more preferably at least 75 mM, such as at least 80 mM. Lower concentrations are possible but generally less effective. Sucrose is a preferred example of saccharide.

In an alternative embodiment the plant material is incubated or at least partly immersed in a medium comprising the antisense oligonucleotide and the at least one saccharide. For instance, plant seeds can be incubated in the medium during plant germination.

When incubating the plant material in the medium, it is generally possible to use somewhat lower concentrations of the antisense oligonucleotide as compared to spraying an aqueous solution onto the plant material. Thus, the medium preferably comprises the antisense oligonucleotide at a concentration of at least 100 μM, more preferably at least 200 μM, such as at least 400 μM. Also concentrations lower than 100 μM can be used. The saccharide concentration in the medium could be as stated above or alternatively at least 50 mM, preferably at least 75 mM, such as at least 100 mM. Lower concentrations are possible but generally less effective.

The embodiments of this aspect can be used in connection with various essential genes that encodes molecules that are essential for survival or viability of the plant material. A particular example of such an essential gene is the gene encoding 5-enolpyruvylshikimate-3-phosphate synthase (EPSPS). This enzyme is involved in the Shikimate pathway of plant aromatic amino acid synthesis. Basically, if its enzyme activity is inhibited, plant cells are killed. This enzyme is also the target of the well-known chemical herbicide ROUNDUP®.

The mutated essential gene is then a mutated form of EPSPS comprising at least two site mutations. The antisense oligonucleotide is then designed to bind to the mRNA transcribed from the wild type EPSPS but not bind to mRNA from the mutated form of EPSPS. Examples of such antinsense oligonucleotides that could be used can be selected among the antisense ODNs presented in SEQ ID NO: 2, SEQ ID NO: 4, SEQ ID NO: 6, SEQ ID NO: 7, SEQ ID NO: 9, SEQ ID NO: 11, SEQ ID NO: 16, SEQ ID NO: 17 or SEQ ID NO: 18 of the attached sequence listing and further identified in the experiments section.

Another example of an essential gene that can be used according to the embodiments to produce the mutated essential gene and the antisense oligonucleotides is the gene encoding 1-deoxy-D-xylulose 5-phosphate reductoisomerase (DXR). DXR is an enzyme that is essential in the metabolism of many important components, such as abscisic acid (ABA), gibberellins, chlorophylls, etc. The mutated essential gene is then a mutated form of DXR comprising at least two site mutations with regard to native form of DXR in the plant material. Suitable antisense oligonucleotides that can be used if DXR is selected as essential gene can be selected from the group consisting of SEQ ID NO: 12 and SEQ ID NO: 13 presented in the attached sequence listing and identified in the experiments section.

Further non-limiting alternatives of essential genes that can be used according to the embodiments can be selected from genes encoding a molecule selected from the group consisting of actin, adenine phosphoribosyl transferase, cyclophilin, eukaryotic elongation factor 1-alpha, eukaryotic initiation factor 1-alpha, eukaryotic initiation factor 4-alpha, farnesyl pyrophosphate isomerase, glyceraldehyde-3-phosphate dehydrogenase, isopentenyl diphsophate isomerase, ribulose 1,5-bisphosphate carboxylase, 18S ribosomal RNA, 25S ribosomal RNA, alpha tubulin, beta tubulin, ubiquitin-conjugating enzyme and ubiquitin.

There are several computer programs available on the market that can be used to design suitable antisense oligonucleotides against selected target genes. Examples of such programs include Primer3, Oligo 7, Lasergene 9.1 and various software used in designing PCR primers.

The plant material that is genetically modified and selected as disclosed herein can be plant material of any plant species, including monocots and dicots. Non-limiting examples of such plant species can be selected from the group consisting of Arabidopsis, petunia, rice, corn, sorghum, soybean, potato, tomato, cassava, barley and tobacco.

Another embodiment of this aspect relates to a transgenic plant material obtainable according to the previously described method. The transgenic plant material thereby comprises a transcriptionally active form of a gene of interest to achieve heterologous gene expression in the transgenic plant material and a transcriptionally active form of a mutated essential gene. This mutated essential gene encodes a molecule that is essential for viability or survival of the plant material and comprises at least two site mutations, preferably at least two silent site mutations, with regard to a native form of the essential gene encoding the molecule and which is naturally present in the plant material. The gene of interest and the mutated essential gene are preferably incorporated into the genome of the plant material. The transgenic plant material additionally comprises the native essential gene. Hence, the transgenic plant material comprises, preferably incorporated into its genome, the gene of interest and two versions of the essential gene, one native or wildtype and one mutated.

A further embodiment of this aspect relates to a binary vector system that can be used to produce transgenic plant material as disclosed herein. The binary vector system comprises a mini-Ti plasmid comprising an origin for replication for Agrobacterium, a gene of interest that is capable of being transcribed in a plant material and a mutated essential gene that is capable of being transcribed in the plant material. The mutated essential gene encodes a molecule that is essential for survival of the plant material and comprises at least two, preferably silent, site mutations with regard to a native form of the essential gene present in the plant material and encoding the molecule. The gene of interest and the mutated essential gene are inserted into a T-DNA region of the mini-Ti plasmid. The molecule encoded by the mutated essential gene preferably has an identical amino acid sequence as the molecule encoded by the native form of the essential gene. The binary vector system further comprises a helper Ti plasmid lacking the T-DNA region but comprises a vir region.

The gene of interest and the mutated essential gene are preferably flanked by a left T-DNA border and a right T-DNA border, see FIG. 9.

The mutated essential gene comprises at least two silent site mutations with regard to the native form of the essential gene and the molecule encoded by the essential gene therefore has an identical amino acid sequence as the molecule encoded by the native form of the essential gene.

Another aspect of the embodiments relates to the use of antisense oligonucleotides as herbicides. Thus, the antisense oligonucleotides disclosed herein and capable of hybridizing to a portion of mRNA transcribed from an essential plant gene can be used as herbicide to kill a particular plant.

The antisense oligonucleotide, when taken up by the plant, binds to mRNA transcribed from the essential plant gene to form a DNA/RNA (or PNA/RNA) hybrid or a RNA/RNA complex. In either case, translation of the mRNA is inhibited with the consequence of no protein or polypeptide expressed from the essential gene or expressed at very low levels. As a consequence and since the gene product of the essential plant gene is necessary for plant survival and viability, plant cells of the plant will start to die, subsequently leading to the death of the complete plant.

The present embodiments achieve two important criteria for an herbicide. Firstly, it is safe, environmentally friendly and biodegradable. Secondly, it is highly selective and specific. The antisense oligonucleotides are composed of naturally occurring material that is readily biodegradable. The antisense oligonucleotides are furthermore highly specific as experiments show herein. Thus, it is sufficient with a mismatch of two nucleotides to escape gene inhibition. Hence, an antisense oligonucleotide of the embodiments having a length of, for instance 15-25 nucleotides, can be designed to be highly plant specific and thereby only cause gene inhibition and plant death in the selected plant species but will not cause any gene inhibition or plant death in other plant species.

The antisense oligonucleotides are therefore, in this aspect, selected to preferably be species-specific and thereby show a mismatch of at least two nucleotides against corresponding or matching gene sequences in other plant species. Such antisense oligonucleotides can be designed according to any of the previously mentioned computer programs once the particular essential gene for the plant species has been selected and is known. Examples of essential plant genes against which the antisense oligonucleotides can be targeted as herbicides can be selected among the previously mentioned examples.

The herbicide of the embodiments can be used to kill various selected plant species. Examples of such plant species include Arabidopsis, petunia, ryegrass, alfalfa, creeping bent grass, moss and wild soybean.

The antisense oligonucleotide is preferably present in an aqueous solution comprising at least one saccharide selected from the group of sucrose, fructose and glucose in addition to the antisense oligonucleotide. The concentration of the antisense oligonucleotide in the aqueous solution is preferably at least 500 μM, preferably at least 700 μM and more preferably at least 1000 μM. Also concentrations lower than 500 μM can be used. The saccharide is preferably sucrose and is preferably present at a concentration of at least 50 mM, preferably at least 75 mM and more preferably at least 80 mM. Lower concentrations are possible but generally less effective.

An embodiment of this aspect relates to a method of killing a plant. The method comprises contacting the plant with an aqueous solution comprising an antisense oligonucleotide capable of hybridizing to a portion of mRNA transcribed from an essential plant gene of the plant. The aqueous solution preferably comprises at least one saccharide and more preferably at least one saccharide selected from a group consisting of sucrose, fructose and glucose.

The contacting step is preferably performed by spraying the plant with the aqueous solution comprising the antisense oligonucleotide and preferably the saccharide selected from sucrose, fructose and glucose. Other ways of contacting the plant with the antisense oligonucleotide are possible as disclosed herein, such as immersing the plant in the aqueous solution and/or growing the plant in a medium comprising the antisense oligonucleotide.

Another embodiment of this aspect defines a herbicide composition comprising an aqueous solution of an antisense oligodeoxynucleotide capable of hybridizing to a portion of mRNA transcribed from an essential plant gene of a plant and at least one saccharide selected from the group of sucrose, fructose and glucose. The saccharide is preferably sucrose.

In a particular embodiment, the herbicide composition is an aqueous solution consisting of at least one antisense oligodeoxynucleotide capable of hybridizing to a portion of mRNA transcribed from an essential plant gene of a plant and at least one saccharide selected from the group of sucrose, fructose and glucose.

### EXPERIMENTS

### Plants and Plant Growth Conditions

Arabidopsis thaliana cv. Colombia-0 was provided by the Arabidopsis Biological Research Centre (ABRC; Ohio State University, Columbus, Ohio, USA). Seeds of tobacco (Nicotiana tabacum) and petunia (Petunia×hybrida), were purchased from Weibulls (Stockholm, Sweden). Rice (Oryza sativa cv. Nippobare) was obtained from Fujian Academy of Agricultural Sciences (Fuzhou, China). The plants were germinated at 22° C. with a light intensity of 125 μmol photons m⁻¹ s⁻¹ under a regime of 16-hours day and 8-hours night. For leaf experiments, plants were grown under the same condition but in pots (12×12×12 cm) with soil and leaves were dissected from the plants and used for ODN experiments.

### Oligodeoxynucleotides

Oligodeoxynucleotides (ODNs) used in experiments were purchased from Invitrogen (Carlsbad, Calif., USA) and are listed in Table 1.

TABLE 1
Oligodeoxynucleotides (ODNs) used in experiments

| Oligodeoxynucleotide name | Sequence (5′ to 3′) | SEQ ID NO: |
|---|---|---|
| At EPSPS sense ODN1 | CTGCTTCTCGCTGCTCT | 1 |
| At EPSPS antisense ODN1 | AGAGCAGCGAGAAGCAG | 2 |
| At EPSPS sense ODN2 | CTTCTGTTTCCACGGCGG | 3 |
| At EPSPS antisense ODN2 | CCGCCGTGGAAACAGAAG | 4 |
| At EPSPS sense ODN3 | CATGCTTGATGCGTTGAA | 5 |
| At EPSPS antisense ODN3 | TTCAACGCATCAAGCATG | 6 |
| At EPSPS antisense ODN4 | TTCCACATTAAGTCCCAA | 7 |
| pe EPSPS sense ODN1 | CATGCTTGGTGCCTTGAA | 8 |
| pe EPSPS antisense ODN1 | TTCAAGGCACCAAGCATG | 9 |
| pe EPSPS sense ODN2 | CATGCTTGGGGCCTTGGA | 10 |
| pe EPSPS antisense ODN2 | TCCAAGGCCCCAAGCATG | 11 |
| DXR antisense ODN1 | GGGTATTTCACATTGTC | 12 |
| DXR antisense ODN2 | GCTTCAATTGCAGCAAC | 13 |

### Germination Experiments

Seeds of the different plants were sterilized before germination according to a protocol described previously (Nalawade et al, 2012). Arabidopsis seeds were treated in addition at 4° C. for 48 hours for vernalization. Plant germination was performed by placing the seeds on a modified Murashige and Skoog (MS) medium (Murashige and Skoog, 1962). The modified MS medium was 10 times diluted content of the original MS medium recipe and supplemented with 100 mM sucrose and solidified with 0.6% agarose. Sterilized seeds were applied to the modified MS medium in Petri dishes (diameter 4 cm). Germination was conducted by incubating the Petri dishes in a growth chamber. For the germination inhibition of Arabidopsis, tobacco and petunia, 400 μM of each ODN was included in the modified MS medium except otherwise indicated. For the germination inhibition of rice, 800 μM to 1.6 mM of ODN was used. In rice germination experiments, most of the rice endosperm parts were removed before germination. The germinated plants were grown for two to four weeks for observation of ODN inhibition.

### Leaf Experiments

About one month old seedlings of Arabidopsis and petunia were used. Healthy leaves were cut and incubated in 80 mM sucrose solution with 700 μM ODN in most cases, otherwise as indicated. Incubation time used to be 48 and 72 hours to observe ODN inhibition.

### Gene Mutation and Molecular Cloning

One of the two Arabidopsis EPSPS genes with GenBank accession no. NM_130093.2 was used for gene mutation in a kit construction. The binding region (nucleotide 416 to 433) of At EPSPS antisense ODN2 in the gene was selected for mutation. Nucleotide mutations in the gene with 0-site, 2-sites or 5-sites were made by synthesizing entirely new mutated genes at Eurofins MWG Operon (Germany). For the different sites of mutations, the sequence was changed as follows with low-case letters indicating the mutated sites: 0-site, 5′-CTTCTGTTTCCACGGCGG-3′ (SEQ ID NO: 3) to 5′-CTTCTGTTTCCACGGCGG-3′ (SEQ ID NO: 3); 2-sites, to 5′-CTTCTaTTTCCgCGGCGG-3′ (SEQ ID NO: 14); 5-sites, to 5′-CTcCTaTTcCCgCGaCGG-3′ (SEQ ID NO: 15). The synthetic genes were firstly cloned in the pBluescript II plasmid then to a binary vector (GWB2) using the Gateway system (Invitrogen).

### Plant Transformation

The resulted binary vectors with the mutated EPSPS were transformed to Agrobacterium tumefaciens strain C58. Arabidopsis thaliana cv. Colombia-0 was transformed using the floral-dip method (Desfeux et al, 2000) at the Arabidopsis transformation platform (http://at-plattformen.slu.se). Transformed plants (T3) were used for different ODN experiments.

### Spaying Experiments

Two ODNs of At EPSPS sense ODN2 (SEQ ID NO: 3) and At EPSPS antisense ODN2 (SEQ ID NO: 4) were used for spraying experiments. The ODNs were dissolved in 80 mM sucrose to a final concentration of 700 μM and sprayed twice to 2 weeks-old seedlings of Arabidopsis and petunia using a perfume sprayer.

### Antisense Oligodeoxynucleotides Against a Plant Essential Gene can Kill Plants in a Sequence-Specific Manner

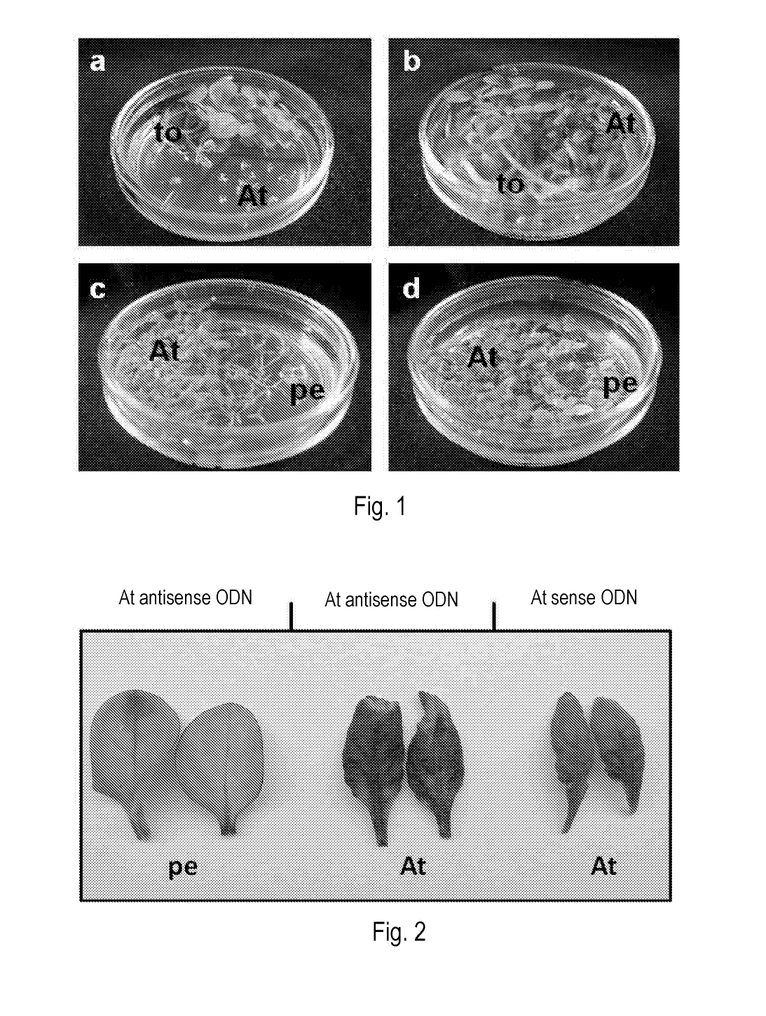

In order to verify whether an antisense ODN against a plant essential gene can kill plants, the plant essential gene called EPSPS was selected. The gene encodes an enzyme named as 5-enolpyruvylshikimate-3-phosphate synthase that is involved in the Shikimate pathway of plant aromatic amino acid synthesis. Basically, if the enzyme activity is inhibited, plant cells are killed. The enzyme is also the target of the well-known chemical herbicide ROUNDUP®. An antisense ODN (At EPSPS antisense ODN3, SEQ ID NO: 6) against Arabidopsis EPSPS and two antisense ODNs (pe EPSPS antisense ODN1, SEQ ID NO: 9; and pe EPSPS antisense ODN2, SEQ ID NO: 11) against petunia EPSPS1 and EPSPS2, respectively, were designed. Sense ODN was used as control (At EPSPS sense ODN3, SEQ ID NO: 5; pe EPSPS sense ODN1, SEQ ID NO: 8; and pe EPSPS sense ODN2, SEQ ID NO: 10). FIG. 1 illustrates that the Arabidopsis antisense ODN could inhibit germination of Arabidopsis but not tobacco. When an ODN mixture of the petunia EPSPS (EPSPS1 and EPSPS2) was applied, germination of petunia was abolished but Arabidopsis was normal. As expected, the corresponding sense ODN did not have any effect on germination of the plants.

Further analysis of the sequence of EPSPS between Arabidopsis (At), tobacco (to) and petunia (pe) revealed that there are two mismatches between the Arabidopsis sequence and tobacco EPSPS1 and EPSPS2, see below. There are also two mismatches and four mismatches between the Arabidopsis sequence and petunia EPSPS1 and EPSPS2, respectively. The results imply that ODN inhibition is exceptionally sequence specific. Two mismatches may be enough to escape inhibition.

| | | |
|---|---|---|
| At EPSPS | CATGCTTGATGCGTTGAA | SEQ ID NO: 3 |
| to EPSPS1 | CATGCTTGGCGCGTTGAA | SEQ ID NO: 16 |
| At EPSPS | CATGCTTGATGCGTTGAA | SEQ ID NO: 3 |
| to EPSPS2 | CATGCTTGGTGCATTGAA | SEQ ID NO: 17 |
| At EPSPS | CATGCTTGATGCGTTGAA | SEQ ID NO: 3 |
| pe EPSPS1 | CATGCTTGGTGCCTTGAA | SEQ ID NO: 8 |
| At EPSPS | CATGCTTGATGCGTTGAA | SEQ ID NO: 3 |
| pe EPSPS2 | CATGCTTGGGGCCTTGGA | SEQ ID NO: 10 |

In order to verify whether the antisense inhibition can be applied to other plant tissues than germinating seeds and other regions of an essential gene besides the one tested above, another antisense ODN (At EPSPS antisense ODN1, SEQ ID NO: 2) was used to test inhibition in leaf tissues. A similar result was achieved in this case. The Arabidopsis antisense ODN caused the “apoptosis-like” symptom in the Arabidopsis leaves after 48 hours incubation but not in the petunia leaves, see FIG. 2. The leaves with the sense ODN control were normal after the incubation. When the ODN binding region in EPSPS was analyzed, again four mismatches were found between EPSPS of Arabidopsis and petunia, see below.

| | | |
|---|---|---|
| At EPSPS | CTGCTTCTCGCTGCTCT | SEQ ID NO: 1 |
| pe EPSPS | CTCCTTCTTGCTGCCTT | SEQ ID NO: 18 |

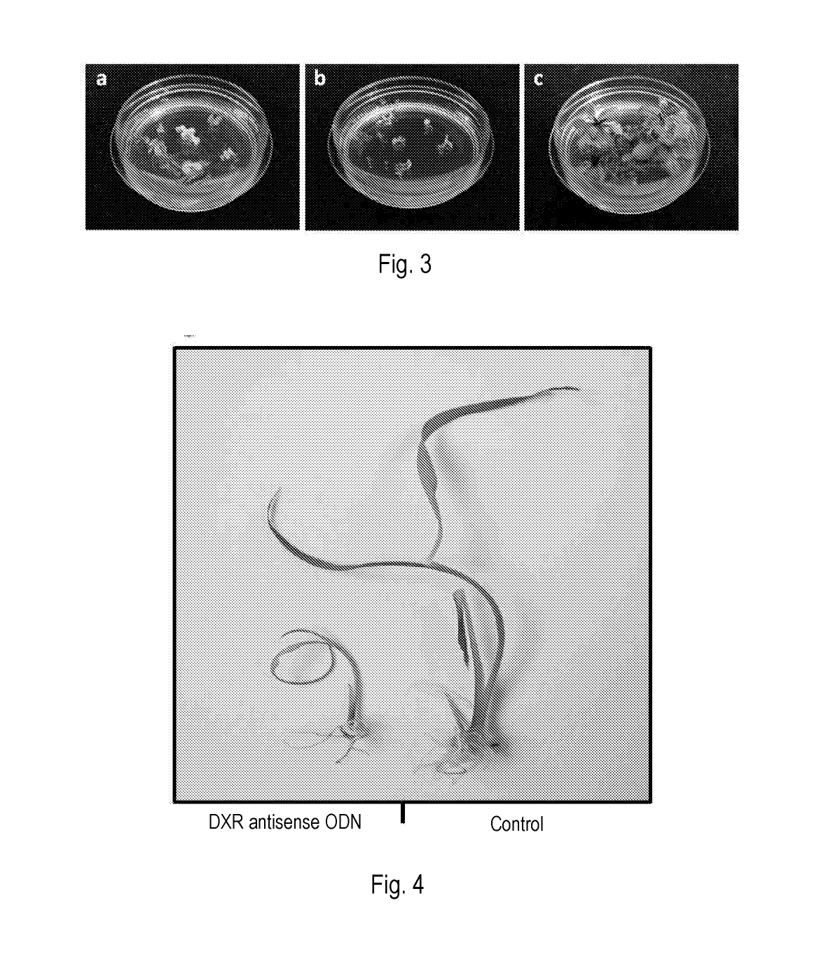

Additional experiments were performed to verify whether antisense inhibition can be applied to monocots in addition to dicots (Arabidopsis and petunia) and for other essential genes than EPSPS. As an example of another plant essential gene, DXR encoding 1-deoxy-D-xylulose 5-phosphate reductoisomerase was selected. DXR is a necessary enzyme in metabolisms of many important components such as ABA (abscisic acid), gibberellins, chlorophylls and etc. For this study, Arabidopsis and rice were used as model plants of dicots and moncots, respectively. FIG. 3 showed that two independent antisense ODNs can inhibit the Arabidopsis germination dramatically. A similar inhibition was also observed in rice germination when DXR antisense ODN1 (SEQ ID NO: 12) was tested, see FIG. 4.

The two antisense ODN sequences used against conserved regions in DXR of Arabidopsis and rice are presented below:

| | | |
|---|---|---|
| DXR antisense ODN1 | GGGTATTTCACATTGTC | SEQ ID NO: 12 |
| DXR antisense ODN2 | GCTTCAATTGCAGCAAC | SEQ ID NO: 13 |

### Application of ODN Inhibition as a Novel Selection System for Transgenic Plants

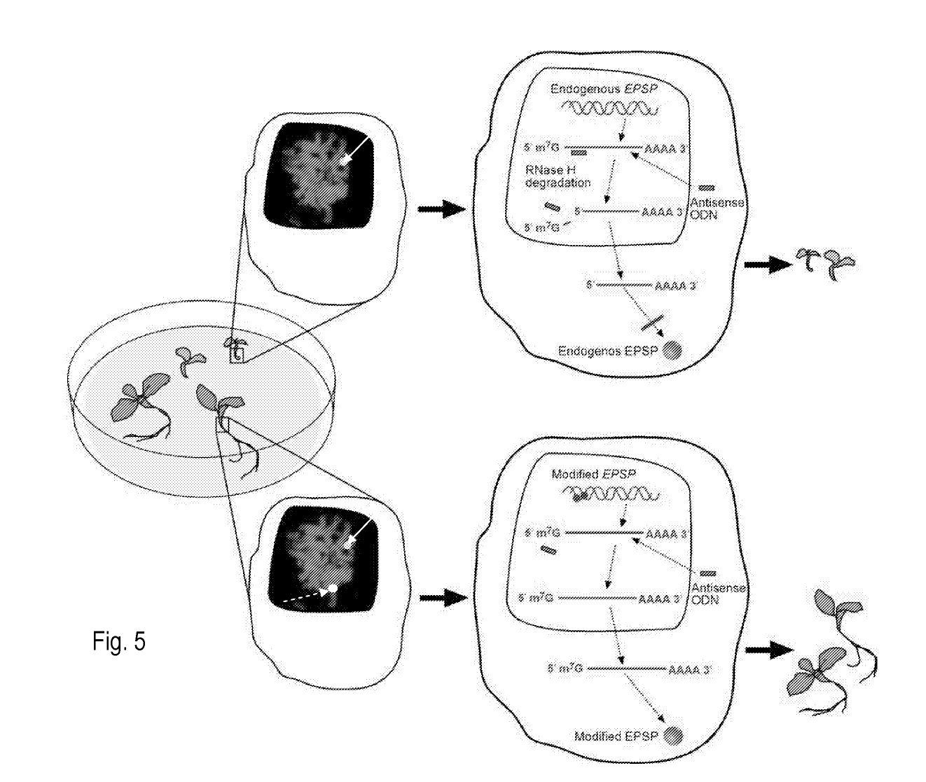

As the previous experiments have indicated the introduction of two nucleotide mutations in an essential gene may help a plant to escape the ODN inhibition, see FIG. 5. Hence, a mutated gene could be used as a marker gene in a totally new-concept selection system using ODNs as selection pressure in transgenic research and applications.

In order to verify the application of antisense ODN inhibition as a selection system, Arabidopsis EPSPS was mutated with 2 or 5 sites and using the non-mutated gene (0-site) as a control. The ODN binding region selected for mutation is based on a leaf experiment where four ODNs were applied and screened for the best inhibition. The four antisense ODNs and the corresponding sense ODN used in this leaf experiment are presented below.

| | | |
|---|---|---|
| At EPSPS sense | CTGCTTCTCGCTGCTCT | SEQ ID NO: 1 |
| At EPSPS antisense | AGAGCAGCGAGAAGCAG | SEQ ID NO: 2 |
| At EPSPS antisense | CCGCCGTGGAAACAGAAG | SEQ ID NO: 4 |
| At EPSPS antisense | TTCAACGCATCAAGCATG | SEQ ID NO: 6 |
| At EPSPS antisense | TTCCACATTAAGTCCCAA | SEQ ID NO: 7 |

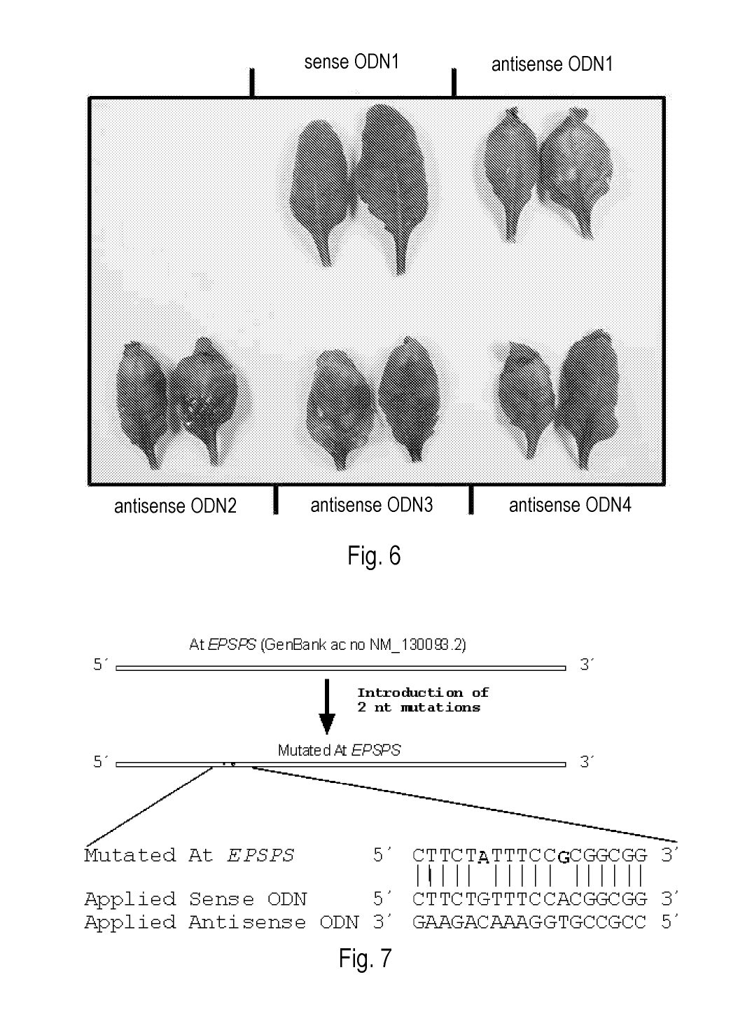

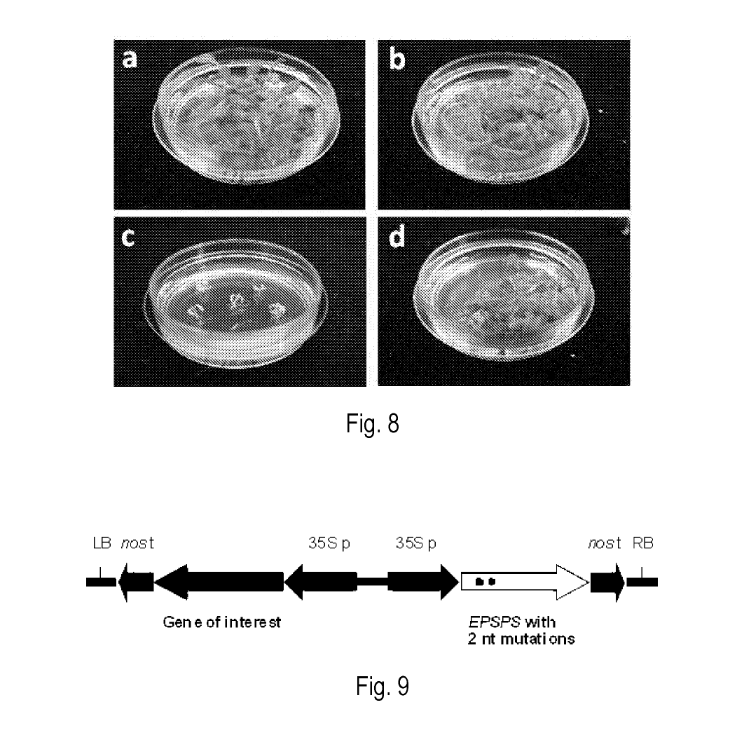

FIG. 6 shows that antisense ODN2 was one of the best ODNs among the tested ODNs. In the antisense ODN binding region of Arabidopsis EPSPS (GenBank accession no. NM_130093.2), 0-site, 2-site and 5-site were chosen for mutations. When the mutated gene was transformed to Arabidopsis, the 2-site mutation resulted in two mismatches with the antisense ODN (At EPSPS antisense ODN2), see FIG. 7. The 2-site mutation was enough to escape the antisense ODN inhibition, see FIG. 8. The results indicate that EPSPS with two site mutations can be used as a selection marker gene in a selection system where antisense ODNs are used as selection pressure.

An example of a product constituting part of a kit for Arabidopsis transformation has been constructed as indicated in FIG. 9.

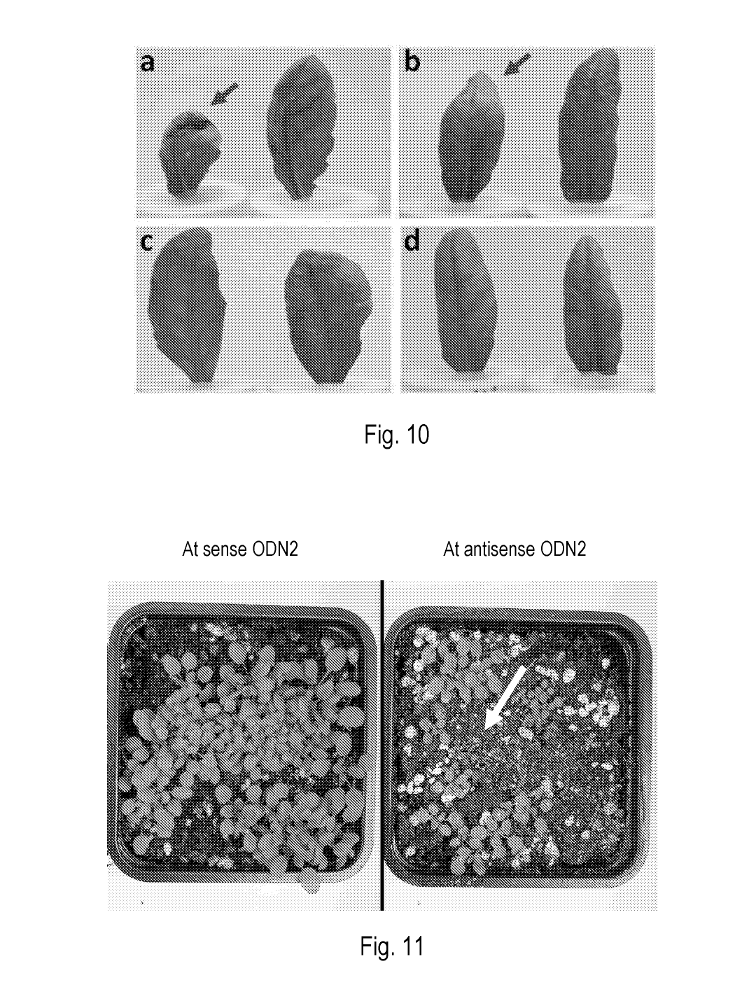

In order to confirm that two mismatches can escape the antisense ODN inhibition in different tissues, leaf experiments were also performed using the transgenic Arabidopsis plants with 0-, 2- or 5-site mutations. A similar result was achieved, see FIG. 10. In the plants of wild type Col-0 and 0-site, the leaves showed symptom of “apoptosis” after 48 hours of incubation with At EPSPS antisense ODN2 (FIG. 10, a and b). However, the leaves of plants with 2- or 5-site mutated EPSPS remained healthy (FIG. 10, c and d).

### Usage of ODNs as Herbicides

Experiments were conducted to confirm that antisense ODNs can be used as potential gene herbicides. ODNs of At EPSPS sense ODN2 and At EPSPS antisense ODN2 where sprayed at a concentration of 700 μM. The Arabidopsis plant growth was inhibited by the antisense ODN, At EPSPS antisense ODN2, and even some of the seedlings were killed, in contrast to the sense ODN control, At EPSPS sense ODN2, see FIG. 11.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

### REFERENCES

Darbani, B., Eimanifar, A., Stewart, C. N., Camargo, W. N. (2007) Methods to produce marker-free transgenic plants. Biotechnol J 2, 83-90.
Desfeux C., Clough S. J., Bent A. F. (2000) Female reproductive tissues are the primary target of Agrobacterium-mediated transformation by the Arabidopsis floral-dip method. Plant Physiol 123, 895-904.
Erikson, O., Hertzberg, M., Näsholm, T. (2004) A conditional marker gene allowing both positive and negative selection in plants. Nat Biotechnol 22, 455-458.
Murashige, T., Skoog, F. (1962) A revised medium for rapid growth and bio assays with tobacco tissue cultures. Physiol Plant 15, 473-97.
Nalawade, S., Nalawade, S., Liu, C., Jansson, C., Sun, C. (2012) Development of an efficient tissue culture after crossing (TCC) system for transgenic improvement of barley as a bioenergy crop. Applied Energy 91, 405-411.
Sonntag, K., Wang, Y., Wallbraun, M. (2004) A transformation method for obtaining marker-free plants based on phsophomannose isomerase. Acta Univ Latv Biol 676, 223-226.
Sun, C., Hoglund, A.-S., Olsson, H, Mangelsen, E., Jansson, C. (2005) Antisense ODN inhibition as a potent strategy in plant biology: Identification of SUSIBA2 as a transcriptional activator in plant sugar signaling. Plant J 44, 128-138.
Sun, C., Ridderstråle, K., Hoglund, A.-S., Larsson, L.-G., Jansson, C. (2007) Sweet delivery—sugar translocators as ports of entry for antisense oligodeoxynucleotides in plant cells. Plant J 52, 1192-1198
Sun, C., Ghebramedhin, H., Hoglund, A.-S., Jansson, C. (2008) Antisense oligodeoxynucleotide inhibition as a potent diagnostic tool for gene function in plant biology Plant Signal Behavior 3, 328-330.

1.-21. (canceled)

22. A method of producing transgenic plant material comprising:
transforming a first plant material with a vector comprising i) a gene of interest that is capable of being transcribed in said first plant material and/or in a second plant material generated from said first plant material and ii) a mutated essential gene that is capable of being transcribed in said first plant material and/or said second plant material, wherein said mutated essential gene encodes a molecule that is essential for survival of said first plant material and/or said second plant material and comprises at least two site mutations with regard to a native form of said essential gene encoding said molecule;
contacting said first plant material or said second plant material with an antisense oligonucleotide capable of hybridizing to a portion of mRNA transcribed from said native form of said essential gene, said portion of mRNA is transcribed from a portion of said essential gene encompassing at least two nucleotides that are site-mutated in said mutated essential gene; and
identifying said first plant material or said second plant material as transgenic plant material capable of transcribing said gene of interest if said first plant material or said second plant material does not show symptoms of gene inhibition with regard to said native form of said essential gene.

23. The method according to claim 22, wherein transforming said plant material comprises:
introducing a mini-Ti plasmid comprising i) an origin for replication for Agrobacterium, and ii) said gene of interest and said mutated essential gene inserted into a T-DNA region of said mini-Ti plasmid into an Agrobacterium cell comprising a helper Ti plasmid lacking said T-DNA region but comprising a vir region; and
transforming said first plant material using said Agrobacterium cell.

24. The method according to claim 22, wherein contacting said first plant material or said second plant material with said antisense oligodeoxynucelotide comprises spraying said first plant material or said second plant material with an aqueous solution comprising said antisense oligodeoxynucelotide and at least one saccharide selected from the group of sucrose, fructose and glucose.

25. The method according to claim 22, wherein contacting said first plant material or said second plant material comprising incubating plant seeds in a medium comprising said antisense oligonucleotide during plant germination and at least one saccharide selected from the group of sucrose, fructose and glucose.

26. The method according to claim 22, wherein identifying said first plant material or said second plant material comprises identifying said first plant material or said second plant material as transgenic plant material capable of transcribing said gene of interest if said first plant material or said second plant material does not show symptoms of cell death.

27. The method according to claim 22, wherein transforming said first plant comprises material transforming said first plant material with said vector comprising said gene of interest and said mutated essential gene encoding 5-enolpyruvylshikimate-3-phosphate synthase.

28. The method according to claim 22, wherein transforming said first plant material comprises transforming said first plant material with said vector comprising said gene of interest and said mutated essential gene encoding a molecule selected from the group consisting of 1-deoxy-D-xylulose 5-phophate reductoisomerase, actin, adenine phosphoribosyl transferase, cyclophilin, eukaryotic elongation factor 1-alpha, eukaryotic initiation factor 1-alpha, eukaryotic initiation factor 4-alpha, farnesyl pyrophosphate isomerase, glyceraldehyde-3-phosphate dehydrogenase, isopentenyl diphsophate isomerase, ribulose 1,5-bisphosphate carboxylase, 18S ribosomal RNA, 25S ribosomal RNA, alpha tubulin, beta tubulin, ubiquitin-conjugating enzyme and ubiquitin.

29. The method according to claim 22, wherein transforming said first plant material comprises transforming plant material of a plant selected from the group consisting of Arabidopsis, petunia, rice, corn, sorghum, soybean, potato, tomato, cassava, barley and tobacco with said vector comprising said gene of interest and said mutated essential gene.

30. The method according to claim 22, wherein said mutated essential gene comprises at least two silent site mutations with regard to said native form of said essential gene and said molecule encoded by said mutated essential gene has an identical amino acid sequence as said molecule encoded by said native form of said essential gene.

31. The method according to claim 22, wherein contacting said first plant material or said second plant material comprises contacting said first plant material or said second plant material with an antisense oligodeoxynucleotide capable of hybridizing to said portion of mRNA transcribed from said native form of said essential gene.

32. The method according to claim 22, wherein contacting said first plant material or said second plant material comprises contacting said first plant material or said second plant material with an antisense oligonucleotide capable of hybridizing to said portion of mRNA transcribed from said native form of said essential gene and having a length of 15 to 25 nucleotides, preferably a length of 15 to 20 nucleotides.

33. A transgenic plant material obtainable according to the method of claim 22.

34. A binary vector system comprising:
a mini-Ti plasmid comprising i) an origin for replication for Agrobacterium, ii) a gene of interest that is capable of being transcribed in a plant material and iii) a mutated essential gene that is capable of being transcribed in said plant material, said mutated essential gene encodes a molecule that is essential for survival of said plant material and comprises at least two silent site mutations with regard to a native form of said essential gene encoding said molecule, said gene of interest and said mutated essential gene are inserted into a T-DNA region of said mini-Ti plasmid, said molecule encoded by said mutated essential gene has an identical amino acid sequence as said molecule encoded by said native form of said essential gene; and
a helper Ti plasmid lacking said T-DNA region but comprising a vir region.

35. The binary vector system according to claim 34, wherein said gene of interest and said mutated essential gene are flanked by a left T-DNA border and a right T-DNA border.

36. A method of killing a plant comprising contacting said plant with an aqueous solution comprising:
an antisense oligonucleotide capable of hybridizing to a portion of mRNA transcribed from an essential plant gene of said plant; and
at least one saccharide selected from the group of sucrose, fructose and glucose.

37. The method according to claim 36, wherein contacting said plant comprises spraying said aqueous solution onto said plant.

38. A herbicide composition comprising an aqueous solution comprising an antisense oligonucleotide capable of hybridizing to a portion of mRNA transcribed from an essential plant gene of a plant and at least one saccharide selected from the group of sucrose, fructose and glucose.

39. The herbicide composition according to claim 38, wherein said at least one saccharide is sucrose.

40. The herbicide composition according to claim 38, wherein said antisense oligonucleotide is present at a concentration of at least 500 μM in said aqueous solution.

41. The herbicide composition according to claim 40, wherein said antisense oligonucleotide is present at a concentration of at least 700 μM in said aqueous solution.

42. The herbicide composition according to claim 41, wherein said antisense oligonucleotide is present at a concentration of at least 1000 μM in said aqueous solution.

43. The herbicide composition according to claim 38, wherein said at least one saccharide is present at a concentration of at least 50 mM in said aqueous solution.

44. The herbicide composition according to claim 43, wherein said at least one saccharide is present at a concentration of at least 75 mM in said aqueous solution.

45. The herbicide composition according to claim 44, wherein said at least one saccharide is present at a concentration of at least 80 mM in said aqueous solution.
